(19) United States
(12) Patent Application Publication
(10) Pub. No.: US 2025/0281309 A1
(43) Pub. Date: Sep. 11, 2025

(54) ENCODING MULTIMODAL INTENSITY AND TEMPORALLY DEPENDENT STIMULATION FOR HAPTIC FEEDBACK

(71) Applicant: BOARD OF TRUSTEES OF THE UNIVERSITY OF ARKANSAS, Little Rock, AR (US)

(72) Inventors: Tommaso Benigni, Chicago, IL (US); Andres Pena, Weston, FL (US); Sathyakumar S. Kuntaegowdanahalli, Bentonville, AR (US); Ranu Jung, Fayetteville, AR (US)

(73) Assignee: BOARD OF TRUSTEES OF THE UNIVERSITY OF ARKANSAS, Little Rock, AR (US)

(21) Appl. No.: 19/073,260

(22) Filed: Mar. 7, 2025

Related U.S. Application Data
(60) Provisional application No. 63/562,625, filed on Mar. 7, 2024.

(51) Int. Cl. A61F 2/72; A61F 2/68; A61N 1/36; G06F 3/01

(52) U.S. Cl. CPC A61F 2/72; A61N 1/36132; A61N 1/3615; A61F 2002/6827; G06F 3/016

(57) ABSTRACT

Embodiments pertain to methods of optimizing peripheral nerve stimulation in a subject by stimulating a peripheral nerve of the subject, where the stimulation includes modulating an intensity and frequency of the stimulation; and receiving sensory feedback from the subject after the stimulation. Additional embodiments pertain to systems and computing devices for optimizing peripheral nerve stimulation in a subject in accordance with the methods of the present disclosure.

### CROSS-REFERENCE TO RELATED APPLICATIONS

This application claims priority to U.S. Provisional Patent Application No. 63/562,625, filed on Mar. 7, 2024. The entirety of the aforementioned application is incorporated herein by reference.

### STATEMENT REGARDING FEDERALLY SPONSORED RESEARCH

This invention was made with government support under W81WXH1910839 awarded by the Department of Defense. The government has certain rights in the invention.

### BACKGROUND

A need exists for more effective systems and methods for optimizing peripheral nerve stimulation. Numerous embodiments of the present disclosure aim to address the aforementioned need.

### SUMMARY

In some embodiments, the present disclosure pertains to methods of optimizing peripheral nerve stimulation in a subject. In some embodiments, such methods include: stimulating a peripheral nerve of the subject, where the stimulation includes modulating an intensity and frequency of the stimulation; and receiving sensory feedback from the subject after the stimulation. In some embodiments, the modulating includes modulating an intensity and frequency of a received percept of the stimulation.

Additional embodiments of the present disclosure pertain to systems for optimizing peripheral nerve stimulation in a subject. In some embodiments, such systems include: a memory; and at least one processor coupled to the memory and configured to implement the following method: stimulating a peripheral nerve of the subject, where the stimulation includes modulating an intensity and frequency of the stimulation; and receiving sensory feedback from the subject after the stimulation. In some embodiments, the modulating includes modulating an intensity and frequency of a received percept of the stimulation.

Further embodiments of the present disclosure pertain to computing devices that include a non-transitory computer-usable medium having computer-readable program code embodied therein. In some embodiments, the computer-readable program code is adapted to be executed to implement a method for optimizing peripheral nerve stimulation in a subject, where the method includes: stimulating a peripheral nerve of the subject, where the stimulation includes modulating an intensity and frequency of the stimulation; and receiving sensory feedback from the subject after the stimulation. In some embodiments, the modulating includes modulating an intensity and frequency of a received percept of the stimulation.

### DRAWINGS

FIG. 1A illustrates a method of optimizing the peripheral nerve stimulation of a subject.

FIG. 1B illustrates a system for optimizing the peripheral nerve stimulation of a subject.

FIG. 2 shows that the surface electrodes are kept stationary on the left arm while the right hand responds. The participant is seated at a table with a screen displaying instructions in front of them. Electrodes are placed on the left arm, which is set to rest on a pad. The right hand is used to manipulate the calibration knob and custom buttons depending on the task.

FIG. 3 shows burst period (BP) modulation with a channel hopping interleaved pulse strategy (CHIPS). This study used charge-balanced, current-controlled biphasic rectangular pulses following the CHIPS strategy to stimulate the peripheral nerve transcutaneously. The number of individual CHIPS pulses for a given intensity level is calculated at the start of every burst duration (BD), along with the length of the BP. The BD remains constant while the interburst interval (IBI) is modulated. The intensity of the percept was modulated by the changing of the charge rate (QR), which is a combination of the pulse amplitude (PA), pulse width (PW) and pulse frequency (PF) inside the individual BDs. Here, QR is represented by the different number of pulses in each BD.

FIGS. 4A-4B show the effect on Weber's ratios of intensity and frequency at different parameter values. FIG. 4A shows a Weber's ratios from the five two alternative forced choice (2AFC) tasks in which participants were asked to differentiate between the intensity of the pair of stimuli while the burst period was set to either a low or high. Low or high QR refers to the QR value of the reference pulse train around which the testing pairs were generated _{BP_L} or _{BP_H} refers to the constant BP value for both pulse trains in the test pair. In the QR continuous set, BP modulation was not performed, and pulses were sent continuously. FIG. 4B shows Weber's ratios from the four 2AFC in which participants were asked to differentiate between the frequency of the pair of stimuli in these tasks. Low or high BP refers to the BP value of the reference pulse train around which the testing pairs were generated. _{QR_L} or _{QR_H} refers to a constant high or low QR value for both pulse trains in the test pair. (**=p<0.005, ***=p<0.0005, ****=P<0.00001).

FIGS. 5A-5B show that the non-modulated dimension has no effect on scalability of intensity or frequency. A min-max method was used to standardize the results for each participant to run statistical tests. FIG. 5A shows the average normalized results of two magnitude estimation task. Participants were asked to rate the intensity of the stimuli, with high and low representing the constant burst period of the stimuli used during that task. FIG. 5B shows results where the participants were asked to rate the perceived frequency of the stimuli, where stimuli with a smaller burst period reported as a higher value and slower being reported as lower, with high and low representing the constant intensity of the stimuli used during that task.

FIG. 6 provides an example of a multidimensional neurostimulation encoding method. Typically, the intensity of the stimulation is continuously modulated with parameters such as pulse frequency, pulse width and pulse amplitude increasing as the perceived intensity increases. In the novel approach, both the intensity and frequency of the percept are modulated. The intensity is modulated in the same way, but only inside a burst duration of constant length while the changing inter-burst interval provides the participant a feeling of changing frequency.

FIG. 7 provides a parameter specification table containing a few constraints that the encoding method in Example 2 requires to function properly and their justification.

FIG. 8 shows an encoding algorithm block diagram used to encode the stimulation in Example 2. The algorithm takes two signal sources: one that will inform the intensity modulation and the second that informs the burst period modulation. The algorithm digitizes the incoming signals and encodes the intensity parameters needed inside the burst duration. Thereafter, the stimulation training is constructed. The algorithm updates constantly in such a way that the stimulation parameters used in the next burst period represent the most up-to-date input signal values.

FIG. 9 shows a parameter compensation approach for more linear intensity percepts. In the conventional approach, a parameter, in this case, amplitude, is kept constant as the charge is modulated to the maximum capabilities of the neurostimulator, creating discreetly perceived intensity levels. In the parameter compensation example, the amplitude is modulated along a narrow range aiming to bridge the discontinuities caused by hardware limitations.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components that includes one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The sense of touch is needed for humans performing motor tasks, ranging from complex activities such as playing the guitar or throwing a ball, to simpler tasks such as holding a cup. The feedback provided by tactile and proprioceptive sensations is desirable for generating force and coordinating hand movements. Haptic feedback technology is being actively researched for enhancing embodiment and performance in the remote control of machinery and surgical equipment, virtual reality, prosthetic technology, and military applications. This is partly because adding haptic feedback to a control system transforms it from an open-loop to a closed-loop system, thereby improving device control.

Various methods exist for providing haptic feedback, including vibrotactile, electrotactile, and pressure cuffs. Although these methods have shown success, they have a few practical limitations. In particular, the aforementioned approaches rely on the mechanoreceptors under the skin to encode stimuli into a perceivable sensation. This can lead to a rapid onset of habituation. The approaches can also cause a reduction in control by necessitating bulky tactors to be placed at the desired stimulation sites. If the stimulation site is on the hand, one of the more sensitive and innervated areas of the body, it might impede the control of joysticks, controllers, and the like.

When feedback matches the location and the quality of the external stimuli, it is more efficiently used and accepted. This matching is possible through the direct stimulation of the peripheral nervous system, also known as peripheral nerve stimulation (PNS). Electric pulses are sent through an electrode placed non-invasively on the skin or invasively around the peripheral nerve. This can be accomplished invasively with cuff electrodes, intrafascicularly with longitudinal intrafasicular electrodes (LIFE) electrodes, or possibly extrafascicularly with transverse intrafascicular multichannel electrode (TIME) electrodes and the Utah Slanted Electrode Array (USEA).

Regardless of the method, electrical signals conveyed through the electrodes generate action potentials in the targeted nerve fibers, which may be perceived as originating from a somatotopically matched area distal to the stimulation site. Through PNS, it is possible to stimulate the nerve anywhere along its track, such as the wrist or elbow, in such a way that sensations are perceived on the hand.

In PNS, whether invasive or not, three parameters of the stimulation pulses are typically modulated to provide a graded sensation: the amplitude of the pulse (PA), the duration of the pulse (PW), and the frequency of the pulse (PF). These can also be combined into a single measure known as the charge rate (QR). These parameters affect the perceived intensity of the stimuli as well as the location, dimension, and quality of the perceived sensation.

Typically, a single PNS stimulation channel only modulates one of these pulse parameters. This creates restrictions on the bandwidth of a single channel to the comfortable ranges of stimulation parameters. One workaround to this has been to develop neurostimulator systems with multiple channels and activation locations that can stimulate various perceivable locations. However, this solution is suboptimal as some activation locations elicit sensations on the same perceived area, and non-invasive electrodes typically create large perceivable regions that are prone to overlap.

As such, a need exists for more effective systems and methods for optimizing peripheral nerve stimulation. Numerous embodiments of the present disclosure aim to address the aforementioned need.

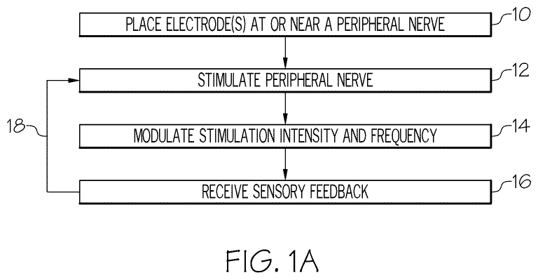

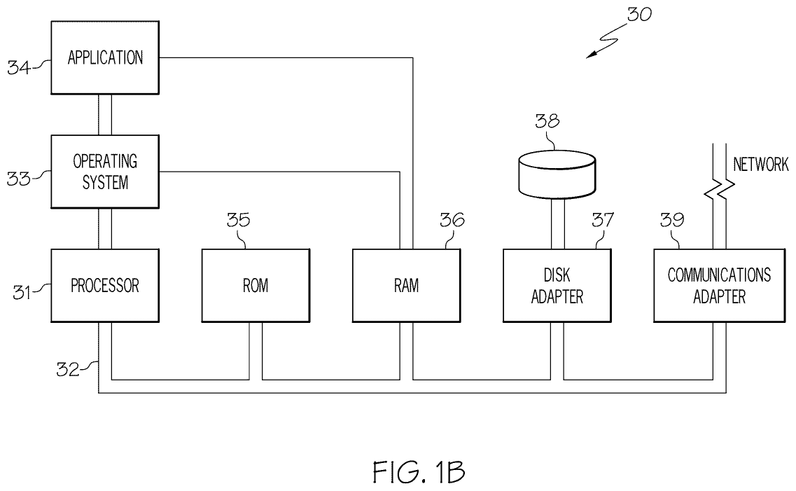

In some embodiments, the present disclosure pertains to methods of optimizing peripheral nerve stimulation in a subject. In some embodiments, such methods include: stimulating a peripheral nerve of the subject, where the stimulation includes modulating an intensity and frequency of the stimulation; and receiving sensory feedback from the subject after the stimulation. In some embodiments illustrated in FIG. 1A, the methods of the present disclosure include: placing electrodes at or near a peripheral nerve of a subject (step 10); stimulating the peripheral nerve of the subject (step 12), where the stimulation includes modulating an intensity and frequency of the stimulation (step 14); and receiving sensory feedback from the subject after the stimulation (step 16). In some embodiments, the methods of the present disclosure are repeated until optimized peripheral nerve stimulation parameters are identified (step 18).

Additional embodiments of the present disclosure pertain to systems for optimizing peripheral nerve stimulation in a subject. In some embodiments, such systems include: a memory; and at least one processor coupled to the memory and configured to implement the following method: stimulating a peripheral nerve of the subject, where the stimulation includes modulating an intensity and frequency of the stimulation; and receiving sensory feedback from the subject after the stimulation.

Further embodiments of the present disclosure pertain to computing devices that include a non-transitory computer-usable medium having computer-readable program code embodied therein. In some embodiments, the computer-readable program code is adapted to be executed to implement a method for optimizing peripheral nerve stimulation in a subject, where the method includes: stimulating a peripheral nerve of the subject, where the stimulation includes modulating an intensity and frequency of the stimulation; and receiving sensory feedback from the subject after the stimulation.

As set forth in more detail herein, the methods, systems and computing devices of the present disclosure can have numerous embodiments. Additionally, the methods, systems and computing devices of the present disclosure can be utilized for numerous applications.

### Peripheral Nerve Stimulation

The methods, systems and computing devices of the present disclosure can stimulate peripheral nerves in various manners. For instance, in some embodiments, peripheral nerve stimulation includes direct stimulation of a peripheral nerve. In some embodiments, peripheral nerve stimulation occurs through a single channel of stimulation.

In some embodiments, peripheral nerve stimulation occurs through one or more electrodes associated with or near a peripheral nerve. In some embodiments, peripheral nerve stimulation occurs through one or more electrodes associated with a skin of a subject.

In some embodiments, the methods of the present disclosure also include a step of placing one or more electrodes at or near a peripheral nerve. In some embodiments, the systems of the present disclosure include one or more electrodes that are operable to be associated with or near a peripheral nerve and provide peripheral nerve stimulation.

### Modulation of Intensity and Frequency of Stimulations

The methods, systems and computing devices of the present disclosure may modulate various intensities and frequencies of stimulations. For instance, in some embodiments, the modulating includes modulating an intensity and frequency of a received percept of a stimulation. In some embodiments, the modulated intensity includes one or more pulse parameters. In some embodiments, the one or more pulse parameters includes, without limitation, pulse width, pulse amplitude, pulse period, pulse duration, pulse charge rate, or combinations thereof. In some embodiments, the modulated intensity includes a pulse charge rate.

In some embodiments, the modulated frequency includes, without limitation, a graded frequency, a pulse frequency, a stimulation burst period, or combinations thereof. In some embodiments, the modulated frequency includes a stimulation burst period. In some embodiments, the stimulation burst period lasts longer than 10 ms. In some embodiments, the stimulation burst period lasts longer than 40 ms. In some embodiments, the stimulation burst period ranges between 10 ms and 50 ms. In some embodiments, the stimulation burst period ranges between 50 ms and 200 ms.

The methods, systems and computing devices of the present disclosure may modulate intensities and frequencies of stimulations in various manners. For instance, in some embodiments, the modulation includes encoding one or more stimulation parameters. In some embodiments, the modulation includes adjusting stimulation current around a reference point.

In some embodiments, the modulation of stimulation includes segmenting the stimulation into on and off cycles. In some embodiments, the modulated frequency includes a stimulation burst period that includes an on time and an off time. In some embodiments, the modulation of the intensity and frequency of a peripheral nerve stimulation includes: (1) segmenting the stimulation into a burst period on time and a burst period off time; (2) modulating the intensity of the stimulation during the burst period on time; and (3) modulating the duration of the burst period off time, burst period on time, or combinations thereof. In some embodiments, the modulating of the intensity and frequency of a peripheral nerve stimulation includes: (1) segmenting the stimulation into a constant burst period on time and a variable burst period off time; (2) modulating the intensity of the stimulation during the burst period on time; and (3) modulating the duration of the burst period off time.

### Receipt of Sensory Feedback

The methods, systems and computing devices of the present disclosure may receive various types of sensory feedback. For instance, in some embodiments, the sensory feedback includes a perceived frequency of a percept and a perceived intensity of a percept. In some embodiments, the sensory feedback is in the form of a graded frequency percept and a graded intensity percept.

In some embodiments, the sensory feedback is in the form of haptic feedback. In some embodiments, sensory feedback is received from one or more sensors. In some embodiments, the systems of the present disclosure also include one or more sensors operable to receive sensory feedback.

### Subjects

The methods, systems and computing devices of the present disclosure may be utilized to optimize peripheral nerve stimulation in various subjects. For instance, in some embodiments, the subject is a human being. In some embodiments, the subject is suffering from a neurological disorder. In some embodiments, the subject is an amputee. In some embodiments, the subject is immersed in a virtual or augmented reality.

### Modes of Operation

The methods, systems and computing devices of the present disclosure may have various modes of operation. For instance, in some embodiments, the methods of the present disclosure are repeated until optimized peripheral nerve stimulation parameters are identified.

In some embodiments, the methods of the present disclosure occur through the use of a peripheral nerve stimulation (PNS) sensory feedback system. In some embodiments, the systems of the present disclosure are in the form of a PNS sensory feedback system.

### Systems and Computing Devices

The systems and computing devices of the present disclosure can include various types of computer-readable storage mediums. For instance, in some embodiments, the computer-readable storage medium can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, or combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions for systems can be downloaded to respective computing/processing devices from a computer-readable storage medium or to an external computer or external storage device via a network, such as the Internet, a local area network (LAN), a wide area network (WAN) and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, Python, or the like, and procedural programming languages, such as the “C” programming language or similar programming languages.

In some embodiments, the computer-readable program instructions may execute entirely on the user's computer, partly on the user's computer, as a stand-alone software package, partly on the user's computer and partly on a remote computer or entirely on the remote computer or server. In the latter scenario, the remote computer may be connected in some embodiments to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure for optimizing peripheral nerve stimulation as discussed herein may be implemented using a system illustrated in FIG. 1B. Referring now to FIG. 1B, FIG. 1B illustrates an embodiment of the present disclosure of the hardware configuration of a system 30 which is representative of a hardware environment for practicing various embodiments of the present disclosure.

System 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for optimizing peripheral nerve stimulation as discussed in the present disclosure, such as in connection with FIGS. 1A, 2-3, 4A-4B, 5A-5B, and 6-9.

Referring again to FIG. 1B, read-only memory (“ROM”) 35 is connected to system bus 32 and includes a basic input/output system (“BIOS”) that controls certain basic functions of system 30. Random access memory (“RAM”) 36 and disk adapter 37 are also connected to system bus 32. It should be noted that software components including operating system 33 and application 34 may be loaded into RAM 36, which may be system's 30 main memory for execution. Disk adapter 37 may be an integrated drive electronics (“IDE”) adapter that communicates with a disk unit 38 (e.g., a disk drive). It is noted that the program for optimizing peripheral nerve stimulation, as discussed in the present disclosure, such as in connection with FIG. 1B, may reside in disk unit 38 or in application 34.

System 30 may further include a communications adapter 39 connected to system bus 32. Communications adapter 39 interconnects system bus 32 with an outside network (e.g., wide area network) to communicate with other devices. In some embodiments, system 30 may also include GPU clusters.

Aspects of the present invention are described herein with reference to flowchart illustrations and/or block diagrams of methods, apparatus (systems), and systems according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other device to cause a series of operational steps to be performed on the computer, other programmable apparatus or other device to produce a computer-aided process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and systems according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Advantages and Applications

The methods, systems and computing devices of the present disclosure can have numerous advantages. For instance, in some embodiments, the methods, systems and computing devices of the present disclosure allow for more precise control and perception of sensations. In some embodiments, the methods, systems and computing devices of the present disclosure allow nerves to feel sensations more realistically, thereby improving the way information is sent to the brain. In some embodiments, the methods, systems and computing devices of the present disclosure allow users to perceive sensations from multiple sensors inside a single channel of stimulation. In some embodiments, the methods, systems and computing devices of the present disclosure allow for the sensations to be felt in or on the hands, thereby making them more natural and easier to interpret. In some embodiments, the methods, systems and computing devices of the present disclosure provide richer and more detailed tactile sensations by tweaking the electrical signals sent to nerves.

As such, the methods, systems and computing devices of the present disclosure can have numerous applications. For instance, in some embodiments, the methods, systems and computing devices of the present disclosure may be used to enhance nerve sensations by the subject. In some embodiments, the methods, systems and computing devices of the present disclosure may be used to enhance sensory feedback from prosthetic limbs. In some embodiments, the methods, systems and computing devices of the present disclosure may be used to enhance sensory feedback from brain computer interfaces (BCI). In some embodiments, the methods, systems and computing devices of the present disclosure may be used to enhance the operation of sensory therapeutic orthoses. In some embodiments, the methods, systems and computing devices of the present disclosure may be used to enhance the effectiveness of neuromodulation therapies (electroceuticals).

In some embodiments, the methods, systems and computing devices of the present disclosure allow for better control and a more natural feel in applications such as virtual reality, prosthetics, and remote-control systems. In some embodiments, the methods, systems and computing devices of the present disclosure may be utilized as therapeutic orthoses for motor and sensory impaired individuals afflicted by stroke or carpal tunnel syndrome (CTS) nerve damage. In some embodiments, the methods, systems and computing devices of the present disclosure may be utilized as electroceuticals to provide neuromodulating therapies to replace the need for drugs for many chronic ill-health conditions.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Simultaneous Modulation of Pulse Charge and Burst Periods in Non-Invasive Peripheral Nerve Stimulation Elicits Two Perceivable and Differentiable Referred Sensations

This Example aimed to investigate the efficacy of a multidimensional stimulation technique in peripheral nerve stimulation (PNS) to provide multiple individually distinguishable percepts through a single stimulation channel. Two modes of sensation were provided simultaneously: intensity conveyed by the modulation of the pulse charge rate (QR) inside short discrete bursts and frequency conveyed by the modulation of the burst period.

Two experiments investigated whether burst periods and their interactions with a charge-rate model of providing intensity percepts could be distinguished. A series of two alternative forced choice tasks (2AFC) was used to investigate burst period modulation's role in intensity discernibility. Magnitude estimation tasks were used to determine any interactions in the scalability of both burst periods and intensity.

Results from the 2AFC revealed that burst periods can be individually discriminated as a gradable frequency percept in PNS. Additionally, the length of burst periods did not significantly influence intensity discrimination when the burst duration was constant. Participants could also produce the same scale of intensity and frequency regardless of the value of the second dimension. This suggests that multidimension encoding is a promising approach for increasing information throughput in sensory feedback systems.

This Example provides valuable insights into haptic feedback through PNS, offering pronounced benefits for virtual reality applications and individuals with upper limb amputations. The feasibility of the multidimensional encoding approach in control tasks opens new avenues for developing enriched sensory feedback systems that foster more sophisticated haptic feedback mechanisms. These findings hold significant potential for groundbreaking research and development in this field.

From complex motor tasks like playing the guitar or throwing a ball to simpler tasks such as holding a cup, the feedback provided by tactile and proprioceptive sensations during the performance of a task is imperative for generating force and overall coordination. This understanding has led to the exploration of haptic feedback to recreate the sense of touch in various applications, such as virtual and augmented reality experiences, controlling machinery remotely, and enhancing the functionality of prosthetics.

For the over two million people with some form of limb loss or other peripheral nerve damage, the loss of sensory function can cause a significant reduction in functional outcomes. Although upper-limb myoelectric prosthesis users might hear the sound of the motors and receive some proprioceptive information based on the devices' and the weight of an object, many cite a lack of haptic sensory feedback as a reason for prosthesis abandonment. Prosthesis abandonment may lead to reduced functional outcomes in both activities of daily living and workplace adjustment, contralateral and chronic pain, and ultimately reduced quality of life.

### Example 1.1. Background

Non-invasive vibro-tactile feedback has been explored in people with upper and lower limb amputations. However, this feedback is prone to habituation and is perceived directly underneath the tactors. Novel methods, such as electrical stimulation to the peripheral nerve targeting sensory fibers, take advantage of existing afferent neural pathways eliciting distally referred somatotopic sensations. As an example, this method can stimulate the median nerve and elicit sensations on the index finger and thumb.

Electrical stimulation is currently being assessed as a means of providing sensory feedback using neural implants. Typically, the intensity of the percept is modulated to provide graded percepts. Modulation of the pulse parameters such as pulse width (PW), pulse amplitude (PA), and pulse frequency (PF), or a combination of all three, has been shown to affect the intensity of the stimulation.

Non-invasive methods of providing sensory feedback utilizing transcutaneous electrical nerve stimulation (TENS) have also been used to target sensory pathways in peripheral nerves to evoke distally referred sensations in areas innervated by those nerves. For example, allowing electrodes at the elbow or wrist to elicit sensations on the hand.

Applicant has shown that intensity modulation using non-invasive methods produce similar results to some neural implants, and can extend the potential uses for somatotopic sensory feedback to a virtual reality environment. Non-invasive stimulation currently does not have the same selectivity as implanted approaches, stimulating whole nerves instead of having fascicular or sub-fascicular selectivity.

The limit of information conveyed through a single stimulation channel is dependent on the range of stimulation parameters within which comfortable intensity percepts are perceived. To overcome this problem, the typical solution has been to increase the number of electrode activation sites that target different populations of nerve fibers, thereby increasing the number of perceivable sensation locations, and has been shown to have functional benefits in case studies. However, this solution is suboptimal because it excludes cases where regardless of the stimulation site, only a single area can be perceived, regardless of the stimulation site on the same nerve or fascicle, such as in some applications of non-invasive PNS or with more severe peripheral nerve damage.

It is, therefore, necessary to find a parameter that might convey functionally relevant information without interfering with the perceived intensity of the stimulation. In vibrotactile stimulation, the most salient temporal feature for vibrotactile frequency was the length of the silent gap between bursts of pulses rather than the frequency of the vibration itself. It was later confirmed that this phenomenon occurred regardless of the stimulated mechanoreceptor. This was shown to be a neurological phenomenon by directly stimulating the nerve through microneurographic stimulation of the fingers. This study suggested that the interburst interval (IBI) might best explain perceived frequency rather than the frequency of pulses or the rate of delivered charges.

To that end, the possibility of modulating the Burst Period (BP) to alter the perceived frequency and encode information in PNS has not been explored. Applicant proposes that changing the BP during stimulation can be perceived as different frequencies within the constrained range necessary to be functionally relevant for prosthesis use. At least three perceivable levels will be seen when asked to scale this range of BPs through a magnitude estimation task. Applicant also proposes that the length of the BP in this constrained range does not affect discriminability, measured by the average Weber's ratio, of the perceived intensity. Instead, a low intensity will reduce discriminability of frequency, significantly increasing the Weber's ratio of BPs, as perceived sensations near threshold are more difficult to detect.

With this Example, Applicant aims to explore novel encoding methods for more advanced and versatile haptic feedback systems, moving closer to a future where technology can offer a touch experience much closer to natural perception. This development could potentially improve virtual reality environments and prosthetic technologies.

### Example 1.2. Able-Bodied Human Subjects

Written informed consent was obtained from twelve participants, six female and six male participants, with an average age of 24.9±4.5 years old. Two participants were left-hand dominant, and ten were right-hand dominant, in compliance with the University of Arkansas Institutional Review Board, which approved this study protocol (IRB #2201379281). All prospective participants were screened prior to the study to determine eligibility. Participants were able-bodied, with no sensory disorders or any self-reported condition listed as a contraindication for transcutaneous electrical stimulation (i.e. pregnancy, epilepsy, lymphedema, or cardiac pacemaker).

### Example 1.3. Experimental Setup

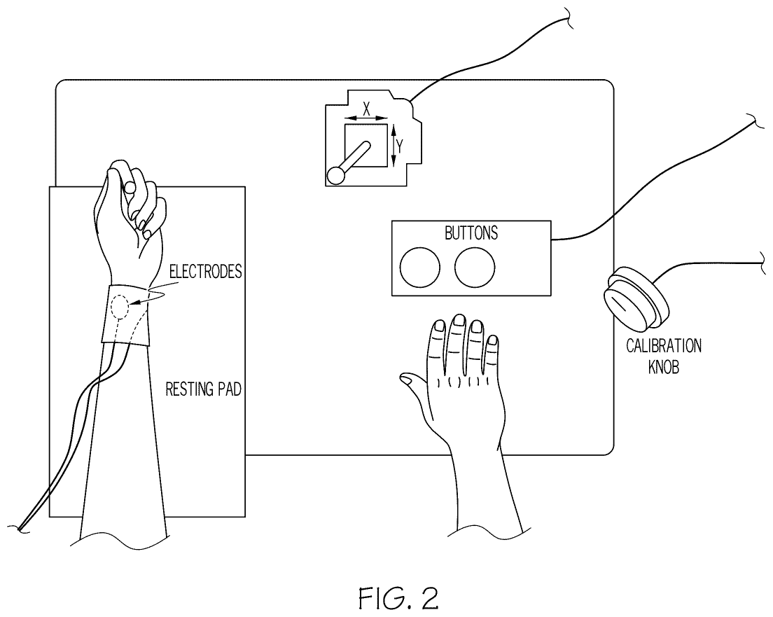

A multi-channel bio-stimulator (TDT IZ2-16H, Tucker-Davis Technologies, Alachua, FL, USA) delivered charge-balanced, current-controlled biphasic rectangular pulses. To minimize local discomfort near the stimulating electrodes, a Channel-Hopping Interleaved Pulse Scheduling (CHIPS) strategy was implemented. This approach used interleaved current pulses to selectively activate underlying nerve fibers without affecting cutaneous receptors. The median nerve was targeted transcutaneously via four small, self-adhesive gel electrodes positioned around the left wrist. These electrodes were carefully adjusted and tested to ensure they elicited distally referred sensations only. To prevent electrode movement during data collection, pre-wrap tape was lightly wrapped around the wrist. Participants were seated at a table facing a display monitor that provided instructions. As illustrated in FIG. 2, they rested their left arm, ulnar side down, on a cushioned support pad and adjusted themselves for comfort. Depending on the specific task, participants used their right hand to either press a custom response button for reporting perceptual responses or turn a control knob to explore different stimulation parameters. Custom experimental control software, written in Python 3, managed the organization and execution of the experiments while storing the collected data.

### Example 1.4. Encoding Approach

Graded intensity percepts were provided to the participants by modulating the pulse frequency (PF) and pulse width (PW) simultaneously in accordance with the charge-rate model. This charge-rate (QR) approach has been shown to elicit a wider range of graded intensity percept than modulating either PF or PW individually. Both PF and PW varied linearly along their respective ranges, while maintaining a constant PA. The range for PF modulation was calibrated according to each participant's ability to detect pulse fusion, the point where pulses begin to feel continuous, and pulse saturation, where intensity levels off. Similarly, the PW modulation range was set between the minimum level that evoked a reliable percept and the maximum level that remained comfortable for the participant. This intensity encoding approach has been used non-invasively to convey a sense of force when grasping virtual objects.

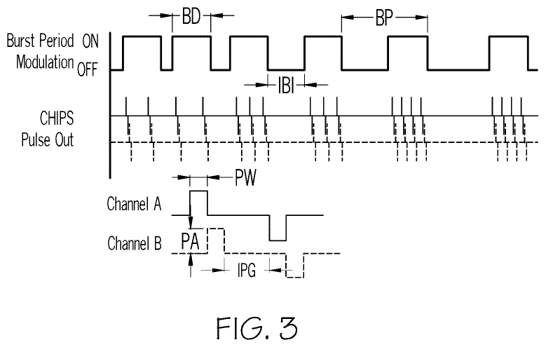

Graded frequency percepts were explored by modulating the burst period (BP) of the stimulation, which consists of the burst duration (BD) when stimulation pulses are actively being sent and a variable interburst interval (IBI) when there is no stimulation, as shown in FIG. 3.

In the experiments, Applicant varied the BP timing between 50 and 200 ms. The burst period is composed of the BD and IBI. Stimulation pulses are sent out during the BD, linking its length to the percept's intensity. To avoid influencing the intensity, the BD needs to remain constant so that only the aforementioned intensity stimulation parameters (PA, PW, PF) affect the intensity. The BD length was determined by two key factors. Firstly, prior research has indicated that when pulse periods drop below 10 ms, the perceived intensity becomes a partially weighted sum of the pulses. Conversely, increasing pulse periods above 15 ms allows participants to distinguish individual pulses. This suggested that the Burst duration must be longer than 10 ms, and that lowest pulse frequency must be around 60 hz.

Secondly, Applicant considered the need for the burst duration to accommodate multiple pulses, as the pulse frequency is known to influence intensity. To validate Applicant's approach, Applicant conducted pilot tests, which showed that a constant burst duration of forty ms enabled a range of discernable intensities to be conveyed within comfortable levels. This translates to a range of 2-8 stimulation pulses within each BD.

The IBI range was set between 10 and 160 ms. The upper limit was determined by the Nyquist rate, the minimum period needed to sample an event, corresponding to the closing speed of a conventional myoelectric prosthesis. The lower limit was established based on the minimum time needed to perceive a gradation in intensity, as well as a noticeable gap between bursts, as identified through microneurography.

### Example 1.5. Experimental Procedures

As previously mentioned, participants were seated in a chair, and four electrodes were placed on their wrists. Then, a participant-controlled calibration was performed to determine the range of parameters that would provide comfortable sensations. Once completed, participants performed nine different two alternative forced-choice tasks and four magnitude estimation tasks. Each of the tasks will be discussed in further detail in the sections below.

### Example 1.6. Calibration

Before performing any experimental tasks, PA, PW, and PF ranges were identified per participant to provide comfortable, reliable sensations. A participant-controlled calibration routine was utilized to find these ranges. To obtain the threshold of perceivable sensations, PA thresholds were collected for five different PWs between 200-700 μs with a one hundred p s step at a low PF (5 Hz). Those results were then fitted to a strength-duration (SD) curve using the Lapicque-Weiss model. Participants were given control of a knob that would increase the PA from 500-2500 μa when rotated clockwise and decrease the PA when rotated counterclockwise. With a chosen PW fixed, they were instructed to find the point where they just began to perceive a sensation and press the response button labeled ‘Next’ to record their response. Each test PW was presented at least twice in random order.

The stimulation PA for the study was then set at 25% above the threshold at a PW of 500 μs. This amplitude at this PW lies above the linear region of the strength duration curve, providing a large range of PWs when modulating pulse widths while also being high enough to provide detectable percepts. A similar method was used to determine the upper and lower limits of the PW and PF range. For the lower limit of the PW, participants were instructed to rotate the same knob, which now modulated between a range of 100-800 μs, until they reliably perceived a sensation; for the upper limit, they were asked to stay below a stimulation value that led to an uncomfortable percept. For the PF limits, participants were instructed to rotate the knob the same knob with a new 10-250 Hz modulation range until they found the lowest possible frequency that was not perceived as pulsating, then to find the level at which the perceived intensity did not change.

Participants took a mid-session break where they could leave the lab space. When they returned, the electrodes were tested to ensure they provided the same percepts as before the break by sweeping through the previously calibrated QR values. If the percept's location moved, and it was distally referred, the entire calibration routine was performed a second time. If, on the other hand, the percept was in the same location but was not perceivable at the previously selected QR threshold, the upper and lower limits of the PW were recalibrated using the same procedures.

### Example 1.7. Two-Alternative Forced-Choice Tasks (2AFC)

To determine the just noticeable difference (JND) between the different burst periods and the effect they might have on the stimulation's intensity, a series of two-alternative forced-choice tasks (2AFC) were conducted. In each task, participants were presented with a pair of equally timed stimulation trains ranging from half a second to one second, with a one-second pause between the bursts. The first train functioned as the reference train and remained constant throughout the set. Participants then compared whether the intensity or frequency of the second stimulation train was the same or different, depending on the dimension being evaluated, by pressing the corresponding buttons in front of them. After the participant's response is recorded, there was a 1.5-second pause before the next pair of stimulation is presented.

For each task, only one parameter was modulated between the pairs: the QR or the BP. Seven unique pairs were evaluated five times (35 presentations of seven unique pairs per task). A unique pair consists of the reference train and a test train. The test train is made of one of seven values, two values above and below the reference value with a step size of less than 25% of the reference parameters, anchoring values with a step size of 50% of the reference parameter, and the reference train. These parameters were chosen to describe the psychometric function in the critical zone while still anchoring them, which was created by fitting the responses to a cumulative distribution. From the distribution function, the point where the stimulation was responded to as being different 75% of the time was recorded as the JND. The Weber's ratio of that value was collected for each trial.

Nine sets of 2AFC tasks were conducted in total, five in which the participants were instructed to focus on the intensity and four in which participants were instructed to focus on the frequency of the pair of stimuli. The reference stimulation for each trial was created using either a high or low (70% and 30% of the range) parameter value depending on what the participants were instructed to focus on, while the other parameter value was set to a constant high or low (70% and 30% of the range) value. Finally, a 2AFC for intensity discrimination at a reference value of 30% was conducted, where the interburst interval was set to zero and stimulation was received continuously.

### Example 1.8. Magnitude Estimation

A magnitude estimation task was performed to understand the scaling of the BP and its interaction with perceived intensity. In this task, participants were asked to rate the stimulation intensity or the frequency of a stimulation train, depending on the task, using the numerical scale. Participants were given an initial stimulation train and asked to verbally assign an arbitrary value to the appropriate aspect of the initial stimulation. Applicant recorded the value and then presented the participant with the second simulation. They were then asked to compare the second simulation to the initial stimulation.

If the second stimulus was perceived as stronger (for intensity) or faster (for frequency), the participants should give a higher number compared to the previous one. For example, if the intensity of the first stimulation train was rated as a ten and the second stimulation was twice as strong, then they were instructed to report a twenty. If it was half as strong, they were instructed to report a five. Participants were always instructed to rate the subsequent stimuli to the previous one, not the first one they received (i.e., B to A, C to B etc.). A score of zero would represent no perceivable sensation. Participant values were then normalized using the min-max method.

A total of four magnitude estimation tasks were performed. Each had equally spaced and randomly selected QR or BP values informed by JND values obtained in pilot studies. As in the 2AFC task for each value, the dimension kept at a constant was set at either a high or low value (70% and 30% of the range, respectively).

### Example 1.9. Randomization

All tasks except the first two were conducted randomly to reduce the effect of training and to introduce the participants to the experiments. The first task was a 2AFC task where the intensity was modulated with a low reference intensity and a constant low BP. The second task was another 2AFC task, where the BP was modulated and the constant QR was low.

### Example 1.10. Statistical Analyses

Before running any statistics, the dataset was checked for normality using a Shapiro-Wilks test. A logistic regression of all the 2AFC tasks assessing whether changes to the BP affected discriminability was used. ANOVAs were used to assess whether there was a significant difference between Weber's ratios. If a significance was detected, a subsequent Tukey's post hoc test was conducted. A k-means clustering analysis was performed to determine the number of differentiable steps detected in the magnitude estimation tasks.

### Example 1.11. Non-Invasive Stimulation with CHIPS Leads to Distally Referred Sensations

Each participant could perceive distally referred sensations in the innervated regions of the median nerve. The proximal palmar, the thenar and parts of the middle and index fingers were the most commonly perceived areas of sensation. None of the participants reported local discomfort or sensations underneath the stimulating electrode after properly fitting the electrodes. The participant-controlled calibration routine was used to fit the stimulating parameters for each participant. With an average stimulating PA of 1996±306 μA, a comfortable PW range of 450-661±81 μs and fusion to saturation. PF range of 62-160±20 Hz. Pre-wrap tape kept the electrodes in the proper position, even during the breaks, with no participants perceiving a different area. Two participants needed to re-adjust their PW range using the calibration routine.

### Example 1.12. Participants can Differentiate Different Values of BP Regardless of the QR

Participants were able to differentiate between different BPs. The results of all the various 2AFC tasks were compiled, and a logistic regression was conducted to assess what aspects of the stimulation would lead a participant to report the second stimulus as different. The regression results showed a large effect size (R²_{Tjur}=0.417). The most significant contributions to the effect size came from differences between the reference QR or BP and the unique pairs parameters (p<0.001) with an odds ratio of 1.14 and 1.08 for QR and BP differences, respectively.

### Example 1.13. BP does not Affect the Discriminability of QR

During the 2AFC tasks where the QR was modulated, participants could distinguish the reference and some of the test stimuli as expected. The collected data was fitted to a cumulative distribution function, and a psychometric curve was obtained. From there, a Weber ratio was extracted for each task.

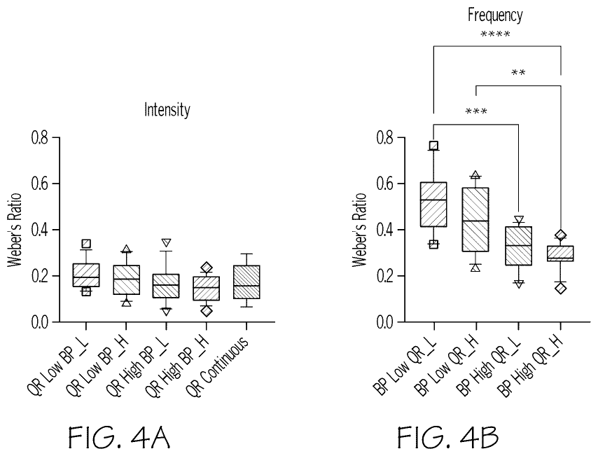

The BP does not affect discrimination of QR as seen in FIGS. 4A-4B. Average ratios for each of the tasks are as follows: 0.205±0.062 (reference QR low and constant BP set to low), 0.185±0.072 (reference QR was low and constant BP set to high), 0.165±0.077 (reference QR was high, and constant BP set to low), and 0.147±0.052 (reference QR was high, and constant BP set to low). An ANOVA was conducted to determine if there was a difference between the ratios. The results showed that there were no significant differences (F=1.29, p=0.314), which seems to suggest that the effect of the BP on the discrimination of intensity is nearly imperceptible (Adjusted R2=0.015). When stimulation was provided continuously, the average Weber ratio for intensity was 0.169±0.08. An ANOVA showed no significant difference from the other intensity ratios, regardless of what value the BP was set to.

### Example 1.14. Increased Intensity Improves the Discriminability of Burst Periods

During the 2AFC tasks where the BP was modulated, participants could again distinguish the reference and comparator stimuli. Average Weber's ratios were 0.531±0.136 (reference BP was low and QR set to low), 0.454±0.143 (reference BP was low and QR set to high), 0.332±0.096 (BP was high, and QR set to low), and 0.287±0.057 (reference BP was high, and QR set to high). The results of an ANOVA warranted a post hoc test (F=11.58, p<0.0001) and a Tukey-Post Hoc was conducted.

Results of the Tukey's lead Applicant to believe that the average Weber's ratio between the reference BP high and intensity high group is different from reference BP low and both intensity high and low groups (p<0.0001, p=0.0005 respectively). Moreover, there was a difference between Weber's ratio between the reference BP High and Low groups with a high-intensity value (p<0.005). The differences among all the other groups were nonsignificant.

### Example 1.15. There are at Least Three Differentiable Values in the BP Scale

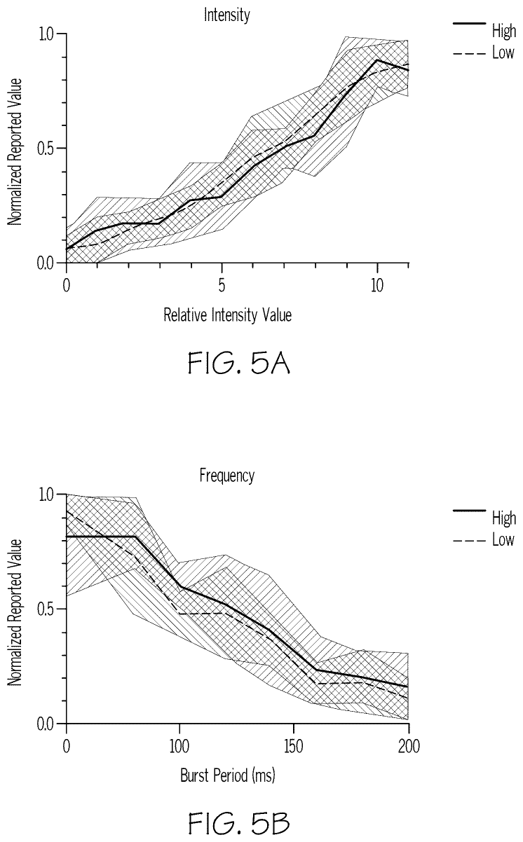

The magnitude estimation tasks were performed, with all participants responding with numerical values greater than zero during stimulation, as shown in FIGS. 5A-5B. A k-means clustering analysis was performed to determine the number of differentiable steps in the scale. The elbow method was then employed to determine the optimal number of clusters with data that scaled the modulation of BP and intensity, showing three differentiable clusters. A two-way ANOVA was conducted to identify if the non-modulated parameter might affect the scaling. The results showed that for intensity and burst duration there was no significant difference in the scale created by the participants.

### Example 1.16. BPs in PNS Convey Tactile Frequency Percepts

The results of this experiment showed that, when stimulating the median nerve non-invasively, perceived frequency can be encoded with burst periods. One mechanism suggested for this frequency percept is that BP stimulation mimics naturally conveyed frequency percepts by following the firing rate of a population of rapidly adaptive (RA) fibers. These fibers begin firing at the start of stimuli, fire at differing rates depending on the intensity of the stimuli, and then abruptly stop firing under constant stimulation.

Although PNS using biphasic pulses currently does not have the specificity to target nerve fiber types, it has been hypothesized that the pattern of spikes overrules the receptor type when conveying frequency. Applicant's study supports the aforementioned hypothesis, as the method of stimulation activated large swaths of nerve fibers and used discrete burst periods conveying a frequency percept.

The short BD during which stimulation could occur was long enough to provide graded-intensity percepts. The results suggest that continuously provided stimulation and stimulation provided through smaller discrete bursts elicit a perceived intensity that can be similarly discriminated. Scaling for the continuous intensity stimulation was not conducted.

### Example 1.17. After the Break, Two Participants Perceived Weak Sensations

Two participants needed their stimulation parameters refitted after a mid-session break. This could have been due to several reasons. The position of the median nerve at the wrist is not constant in relation to the skin. Flexion and extension of the wrist can both cause significant stretching, changing the shape and position of the nerve, both of which can affect the perceived sensation. It is conceivable that participants rested their arms in a different position when resuming the session. It is also possible that as participants were not monitored during their breaks, changes to the electrodermal activity due to excessive movement, or other autonomic changes may have occurred. It is also possible that habituation to the stimulation may have taken place especially as most breaks took place over an hour into the session. Regardless, after recalibration, participants could perform the given tasks without any additional difficulty.

### Example 1.18. The Perceived Frequency Conveyed by BPs was Discriminable after the First Presentation

The second task participants performed during the session was a 2AFC where BPs were modulated. Participants could readily discriminate between different frequencies, especially as the difference in BP increased. There is evidence to suggest that detecting the changes in the perceived tactile frequency is much less of an intuitive task compared to detecting changes in intensity. Changes in sensory processing have been seen during long-term vibro-tactor stimulation training.

Some participants described being very introspective when focusing on the BP, with one participant stating he needed to be “mindful,” which speaks to the novelty of being asked to differentiate frequency. Nevertheless, participants could differentiate between BPs immediately after the task began. It is probable that after extensive training (>30 days), participants would be able to distinguish between these perceived frequency values with much more precision, making it a viable candidate as a dimension for conveying information through sensory feedback.

### Example 1.19. Detecting Frequency is More Difficult when the Percept Intensity is Low

Although stimulation intensity seems to interact with BPs, results of the ANOVA indicate that burst duration is still differentiable at most levels of intensity. Intensity levels at or below thresholds may negate the perceptibility of tactile frequency, as it would be difficult to detect a weak vibro-tactor as implied in the name. However, this Example did not seek to characterize the effect the intensity had on the discrimination of burst duration. Instead, this Example was designed to identify if any interactions existed. The significant difference in Weber's ratio between the slower and faster BP, regardless of the intensity, seems to suggest that there could be a small constant value that needs to be added to the Weber's ratio for BP which is outside the scope of this Example.

### Example 1.20. BP's Scalability Suggests an Application for Transmitting Haptic Feedback

The scaling of the BP collected with the magnitude estimation task shows that at least three values are perceivably different in this reduced BP range. This suggests the possibility of using BPs within this range to transmit relevant sensory feedback. This is especially true for neuroprosthetics applications, as many object discrimination tasks performed to assess neurostimulation in upper limb prostheses use three levels of hardness or compliance.

### Example 1.21. Burst Duration Control

The current procedure for determining the number of pulses per burst duration is to calculate the number by dividing the pulse period for a given intensity value and then rounding that value to an integer. Then, the rising edge of the burst duration and the initial pulse are matched, leading to situations in which the final pulse's falling edge occurs up to seven milliseconds before the falling edge of the burst with the pulse frequency range used for this Example. One mitigation strategy could be shifting the range of usable PFs to ˜100 Hz, reducing the trailing edge of pulses. Another possibility could be adjusting the burst period over a small range to better fit the intensity conveyed. The effects this might have on the discriminability of BP has yet to be investigated.

Electrical pulses sent to the peripheral nerves begin to fuse into a continuous sensation at a pulse frequency of 67-100 Hz. In this Example, the BP was modulated to convey frequencies between 5-20 Hz to convey perceivable gradable intensities. While the lower end of the burst frequency could be fitted to the specific application, the upper end has a much more constrained range of values as multiple individual pulses must be delivered for graded intensity percepts. It is possible to slightly increase this range by moving from a continuous stimulation (IBI=Oms) to discrete bursts, which could increase the range to the point of pulse fusion. However, stimulation hardware with finer temporal resolution would be needed to assess the feasibility of this approach.

### Example 1.22. Implications

In this Example, stimulation pulses were sent in a periodic manner matching the timing of the pulse frequency for a given QR. As discussed previously, similar intensity encoding strategies seem to hold true whether using an invasive or non-invasive stimulation approach. Assessing whether this temporal encoding works with inter or extra-neural electrodes has yet to be performed. It might be possible that this temporal encoding approach produces a different modality of perceived sensation using implanted electrodes, as there is some evidence that temporally modulating the stimulation improve functional outcomes and changes modality of the sensation.

The lack of interaction between perceivable intensity and differing BPs suggests the possibility of multidimensional sensory feedback through a single stimulation channel. Results indicate that participants might perceive this encoding approach as two separate dimensions with the caveat that when the intensity is low, it will be difficult to perceive the mode associated with the BP. For example, if intensity is linked to the value of a force sensor and hand aperture is linked to the burst period, it will be difficult to determine the object's size when lightly grasping it.

### Example 1.23. Conclusions

In sum, this Example investigated whether BPs could effectively encode a secondary dimension, namely, flutter frequency, without simultaneously affecting the perceived intensity. Applicant first showed that BPs can be differentiated as different frequencies inside a range of values that would be functionally relevant for people with an upper limb amputation. Applicant also showed that intensity could be differentiated and scaled without significant differences, regardless of the length of the BP. Results also indicated that any application of this encoding approach in a sensory feedback system would need to consider the initial intensity of the system, as setting intensity to an appropriate level will improve the discriminability of the perceived frequency. Providing independent frequency percepts is an additional step closer to a more complete haptic feedback experience. It could potentially revolutionize virtual reality environments and prosthetic technologies, offering a richer and more natural sensory experience through non-invasive means.

### Example 2. Methods for Encoding Multidimensional Neurostimulation for Haptic Feedback

This Example describes a novel method for encoding stimulation parameters of a peripheral nerve stimulation (PNS) sensory feedback system to increase the information content that can be transmitted to a person to offer both a graded intensity percept and a graded frequency percept. In particular, this Example presents methods for encoding stimulation to increase the amount of information that can be perceived through a single PNS channel. This is achieved by modulating both the perceived intensity of the percept through the modulation of the pulse parameters and the perceived frequency by simultaneously modulating the period of the burst of stimulation.

Additionally, this Example improves upon the conventional encoding approaches, in which only the stimulation pulse parameters are modulated conveying a changing intensity percept, in two ways: the first is by adding a graded frequency percept, by segmenting the stimulation into a constant “on” time and a variable “off” time on top of the conventional parameter modulation. Thus, providing the person receiving the stimulation with a perceivable sensation of both intensity and frequency. This is achieved by changing the pulse parameters to modulate the intensity of the percept while the stimulation is “on” and then changing the duration of the “off” time to modulate the perceived frequency, increasing the total information content that can be transmitted through a single channel.

The second strategy is to improve the grade intensity percept by a method that overcomes digital computation limitations to conveying intensity percepts with PNS. Stimulation pulses are extremely short, lasting only hundreds of microseconds. This means that the resolution of those pulses is limited by the computing speed of the processor being used. By slightly adjusting the stimulation current around a reference point, Applicant compensates for that limitation and creates a finer resolution of stimulation, resulting in a more continuous intensity percept.

Together, these two approaches for encoding stimulation offer a novel method to provide multidimensional information through PNS. This can be used to aid in the performance in tasks like object and texture discrimination that need more information than can be currently provided by a single channel.

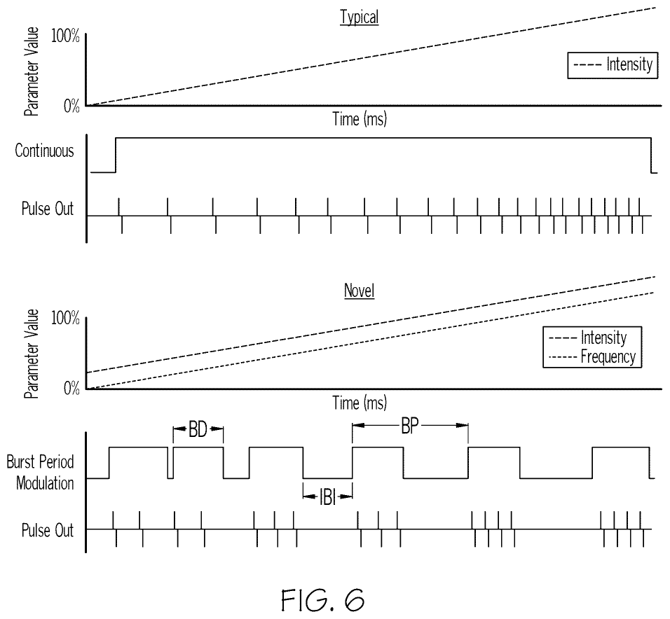

This Example outlines a method for encoding stimulation parameters to simultaneously deliver two modes of perceived sensation, intensity and frequency, through a single channel of haptic feedback through PNS, thereby allowing two sources of information to be conveyed independently. This was accomplished by temporally splitting the stimulation into bursts of pulses with a variable off time between the bursts so that the intensity of the percept is conveyed by the pulse parameters inside the burst, and the frequency by the length of the off time of the burst as seen in FIG. 6. This Example also contains methods to overcome digital computation limitations that create a low-resolution intensity gradation.

### Example 2.1. Multidimensional Encoding Description

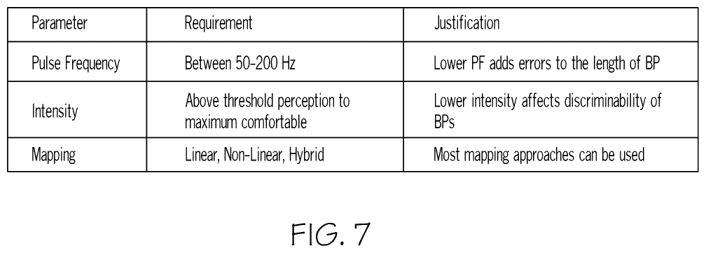

As previously stated, this Example conveys two information sources to a participant through a single channel of PNS. In this section, Applicant first explains which stimulation parameters are modulated to convey each source separately, and how those two sources are integrated into a single output. FIG. 7 outlines the stimulation parameters.

### Example 2.1.1. Modulation of Primary Information Source

The primary information source is conveyed by modulating the stimulation pulse parameters in such a way that the intensity of the percept is changed. Modulation of the pulse amplitude, duration or frequency can all be used to change the intensity of a percept. A combination of all of the above can also be used, offering a more linear gradation of intensity. These pulses are used in peripheral nerve stimulation (PNS) to stimulate the nerve. Typically, these pulses are biphasic, but not necessarily. Biphasic pulses stimulate without leaving residual charge in the surrounding tissue or at the electrode. The pulse charge (PQ), or the number of ions transmitted to the nerve, is equal to the area underneath the pulse calculated as a multiple of the current and time, as set forth in Equation 1.

$$PQ = PA * PW \qquad Equation 1$$

In Equation 1, PA represents the pulse amplitude, or the height of the pulse, in microamps (μA), and PW represents the width or duration of the pulse in microseconds (μs). The pulse charge is indicative of the number of ions moved, which, in turn, is a determinant of the biological response. This can include the number of nerves recruited and the intensity of the stimulation. By changing the pulse frequency (PF), Applicant also reduces the proximity between one pulse and the next pulse. After approximately 60 Hz, they reach a fusion point where the pulses are perceived as arriving continuously and they too affect the intensity of the stimulation.

### Example 2.1.2. Modulation of Secondary Information Source

The secondary information source is conveyed using a temporal encoding approach, giving the user a perception of frequency without affecting the discriminability of perceived intensity. This is accomplished by splitting the stimulation into discreet bursts during which pulses are sent out. We call this method “burst period modulation.” The burst period (BP) includes a constant burst duration (BD) when stimulation pulses are actively being sent, and a variable interburst interval (IBI) when there is no stimulation.

Stimulation pulses are sent out during the burst duration, making its length intrinsically linked to the intensity of the percept. In order not to influence the intensity, the burst duration needs to remain constant so that only the aforementioned intensity stimulation parameters (PA, PW, PF) affect the intensity. The burst duration length was determined by two key factors. First, it needed to be longer than 10 ms, as prior research indicated that pulse periods below 10 ms begin to affect the intensity of the percept. Secondly, Applicant considered the need for the burst duration to accommodate multiple pulses, as this is also known to influence intensity. Testing showed that a constant burst duration of 40 ms enabled a range of discernable intensities to be conveyed within comfortable levels.

As intensity is conveyed during the burst duration, frequency is conveyed during the variable inter-burst interval or the off time. The lower limit of the inter-burst interval is set to 10 ms, which is when gaps are noticed between bursts of stimulation, as determined by microneurography. The upper limit is application-specific. For instance, a haptic feedback system for upper-limb prosthesis users would be set according to the Nyquist ratio, half the time, of the closing time of a typical myoelectric prosthesis or approximately 200 ms.

The exact ranges for burst period modulation depend highly on the specific application and stimulation method employed. One common consideration is the possible interactions between PF and the interburst interval. As PF falls below the fusion point, where the pulses are perceived continuously (approximately 60 Hz), only one or two pulses can fit inside a single burst duration. The distance between the pulses can sometimes be larger than the distance between bursts and can dominate the perception of frequency. This invention therefore only works with PF ranges from fusion to saturation, or the point where increasing PF doesn't affect the percept.

### Example 2.1.3. Integration of Two Sources

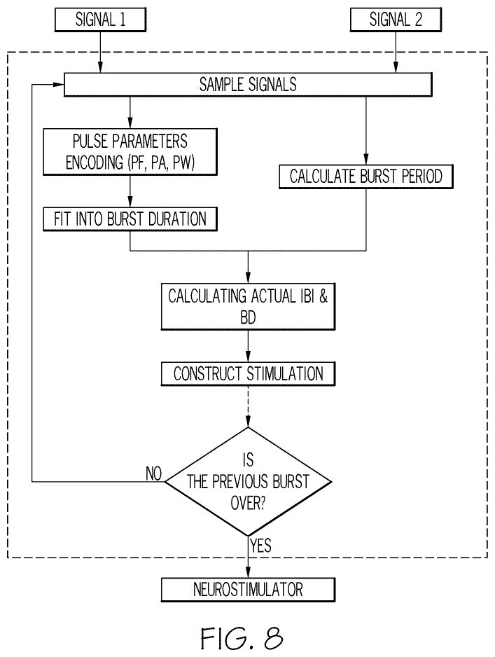

The encoding algorithm that integrates the two information sources can be broken into three functional blocks which are constantly looped, as seen in FIG. 8. The first block is signal recording, in which the external signals that Applicant desires feedback for are sampled. Both digital and analog signals can be recorded, digitized and conveyed. As this encoding strategy encodes two pieces of information, at least two signal sources are needed.

The second block is scheduling the stimulation pulses. The first sampled signal value from the first block, are mapped, or linked to, their fitted pulse parameter (PA, PW, PF) ranges. The shape of stimulation pulse is then stored for the duration of the loop. The second sampled signal is mapped to a burst period length. After the length of the burst period is determined, the number of pulses that fit inside the burst duration is calculated. To ensure that burst periods are delivered accurately, the rising edge of the first pulse is set to coincide with the rising edge of the burst duration.

The third block keeps track of the delivered bursts and sends the stimulation pulses through the neurostimulator when the preceding burst period has ended. It ensures delivery of the bursts as scheduled, eliciting the desired sensations.

The first two blocks of the algorithm constantly update so the most recently sampled signals may be encoded at a frequency of at least 1 Khz. This keeps the delay between sampled sources and the output of the stimulation constant and independent of the length of the burst period.

### Example 2.1.4. Parameter Compensation Method

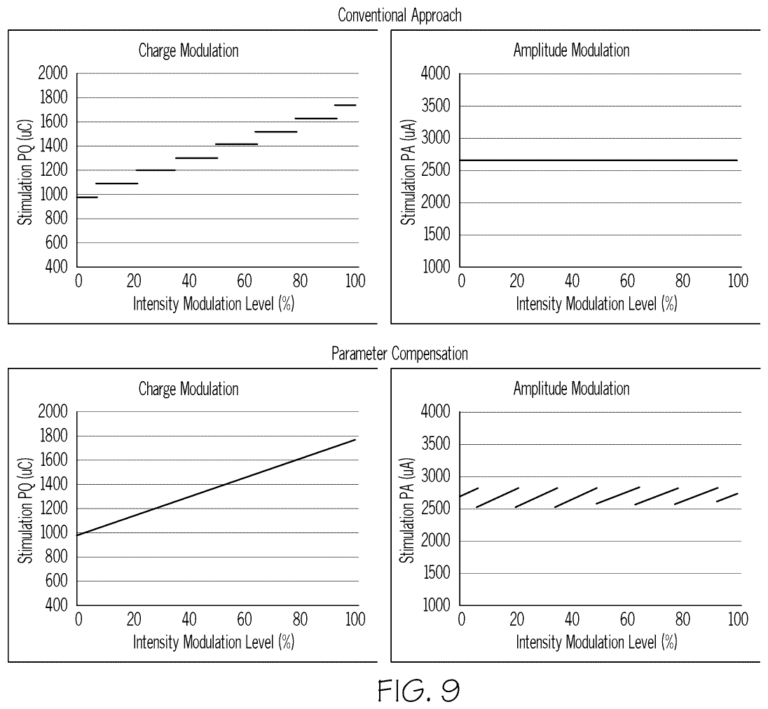

Typically, the gradation of the intensity percept, that is, the number of distinct intensity levels, is limited by the speed of the processor calculating the stimulation pulses. In PNS, pulses are typically short, on the order of hundreds of microseconds. Limitations in computational speeds can create perceivable discontinuities, or steps, in intensity levels. For example, participants will typically have a comfortable range of pulse durations between 450-700 μs. If the processor has a sampling rate of 20 kHz, meaning that the smallest step in PW would be 50 μs, only five possible intensity values could be sent to the participant, as seen in FIG. 9.

This Example introduces a “parameter compensation” method to compensate for sampling rate limitations in available digital computing hardware. Using this method, Applicant perform fine adjustments to PA within a narrow range (about a reference PA associated with each PW) to continuously increase PQ (without gaps or jumps), reducing the perceivable discontinuities in the stimulation intensity. Depending on the need, PQ can be modulated linearly, exponentially, or through a hybrid model.

The smoothing of the pulse parameter discontinuities has two benefits. The first is an increased resolution and increased gradation to the modulation of the first information stream. The second benefit is to remove any interaction these discontinuities may have with the differentiation of the burst periods of the second information stream. The concept can be described by Equations 2 and 3.

$${PQ}_{continuous} = {PW}_{discrete} * ( {PA}_{track} + {PA}_{adj} ) \qquad Equation 2$$

$${PA}_{adj} = ( {PQ}_{continuous} / {PW}_{discrete} ) - {PA}_{track} \qquad Equation 3$$

In Equation 2, PQ_{continuous} is the main parameter being controlled (from PQₘᵢₙ to PQₘₐₓ), PW_{discrete} is the secondary parameter being controlled (from PWₘᵢₙ to PQₘₐₓ). For every PW_{discrete}, PA is adjusted within a narrow range (about PA_{track}) to produce a continuous increase in PQ.

### Example 2.2. Proof of Concept

The approach in this Example was used to increase the bandwidth of sensory feedback a single channel of non-invasive peripheral nerve stimulation could provide. Briefly, two questions of importance were answered in this Example. First, does the burst period modulation affect the perception of intensity? Secondly, does this multidimensional stimulation approach increase the information content that can be sent to a participant?

A multi-channel bio-stimulator (TDT IZ2-16H, Tucker-Davis Technologies, Alachua, FL USA) delivered charge-balanced, current-controlled biphasic rectangular pulses. To answer the first question, 12 participants were used to determine whether burst period modulation affects the discriminability of intensity. Applicant hypothesized that there would be no effect on discriminability. To test this hypothesis, a series of two alternative forced choice tests (2AFC) where the participants were instructed to focus on the intensity of the pair of stimuli were conducted. The reference stimulation for each trial was created using either high or low (70% or 30% of the range) intensity values, while the burst period was set at (70% or 30% of the range) values. A final 2AFC for intensity discrimination at a reference value of 30% was conducted, where the pulses were received continuously.

Results show that the burst period does not affect discrimination of intensity. During the 2AFC tasks where the intensity was modulated, participants could distinguish the reference and some comparator stimuli as expected at all conditions provided. Results were fitted to a cumulative distribution function and a psychometric curve was obtained. From there, a Weber ratio was extracted for each task. An ANOVA was conducted to determine if there was a difference between the Weber's ratios when the burst period was set to either a fast or slow frequency and resulted in a non-significant F-value (F=1.29, p=0.314), which seems to suggest that the effect of the burst period on the discrimination of intensity is nearly imperceptible (Adjusted R2=0.015). When stimulation was provided continuously, the average Weber ratio for intensity was also not significantly different from the other intensity ratios.

To answer the second question, whether multidimensional encoding increases the information content that can be sent to a participant through a single channel of stimulation, Applicant hypothesized that there would be a significant increase in the multidimensional approach compared to the modulation of intensity or burst periods alone. Seventeen participants performed a series of discrete matching tasks in which participants had to match a constant 1.5 s stimulation with a target on a screen. Three iterations of this task were conducted, one where only intensity was modulated, one where only burst periods were modulated, and a third multidimensional approach where both burst period and intensity were changed. Results show that the multidimensional approach had significantly more information than either intensity or burst period modulation alone.

### Example 2.3. Summary

Examples 1 and 2 describe a novel method for encoding stimulation parameters of a peripheral nerve stimulation (PNS) sensory feedback system to increase the information content that can be transmitted to a person to offer both a graded intensity percept and a graded frequency percept. The technology improves upon the conventional encoding approaches, in which only the stimulation pulse parameters are modulated conveying a changing intensity percept, in two ways.

The first improvement is by adding a graded frequency percept, by segmenting the stimulation into a constant “on” time and a variable “off” time on top of the conventional parameter modulation, thus providing the person receiving the stimulation with a perceivable sensation of both intensity and frequency. This is achieved by changing the pulse parameters to modulate the intensity of the percept while the stimulation is “on” and then changing the duration of the “off” time to modulate the perceived frequency, thereby increasing the total information content that can be transmitted through a single channel.

The second improvement is through grade intensity percept by a method that overcomes digital computation limitations to conveying intensity percepts with PNS. Stimulation pulses are extremely short, lasting only hundreds of microseconds. This means that the resolution of those pulses is limited by the computing speed of the processor being used. By slightly adjusting the stimulation current around a reference point, Applicant compensates for that limitation and creates a finer resolution of stimulation, resulting in a more continuous intensity percept. Together, these two approaches for encoding stimulation offer a novel method to provide multidimensional information through PNS. This can be used to aid in the performance in tasks like object and texture discrimination that need more information than can be currently provided by a single channel.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A method of optimizing peripheral nerve stimulation in a subject, said method comprising:
stimulating a peripheral nerve of the subject, wherein the stimulation comprises modulating an intensity and frequency of the stimulation; and
receiving sensory feedback from the subject after the stimulation.

2. The method of claim 1, wherein the modulating comprises modulating an intensity and frequency of a received percept of the stimulation.

3. The method of claim 1, wherein the peripheral nerve stimulation occurs through a single channel of stimulation.

4. The method of claim 1, wherein the peripheral nerve stimulation occurs through one or more electrodes associated with or near the peripheral nerve.

5. The method of claim 4, further comprising a step of placing one or more electrodes at or near the peripheral nerve.

6. The method of claim 1, wherein the modulated intensity comprises one or more pulse parameters selected from the group consisting of pulse width, pulse amplitude, pulse period, pulse duration, pulse charge rate, or combinations thereof.

7. The method of claim 1, wherein the modulated intensity comprises a pulse charge rate.

8. The method of claim 1, wherein the modulated frequency is selected from the group consisting of a graded frequency, a pulse frequency, a stimulation burst period, or combinations thereof.

9. The method of claim 1, wherein the modulated frequency comprises a stimulation burst period.

10. The method of claim 1, wherein the modulating of the stimulation comprises encoding one or more stimulation parameters.

11. The method of claim 1, wherein the modulating of the stimulation comprises adjusting stimulation current around a reference point.

12. The method of claim 1, wherein the modulating of the stimulation comprises segmenting the stimulation into on and off cycles.

13. The method of claim 12, wherein the modulated frequency comprises a stimulation burst period comprising an on time and an off time.

14. The method of claim 13, wherein the modulating of the intensity and frequency of the peripheral nerve stimulation comprises:
segmenting the stimulation into a burst period on time and a burst period off time;
modulating the intensity of the stimulation during the burst period on time; and
modulating the duration of the burst period off time, burst period on time, or combinations thereof.

15. The method of claim 13, wherein the modulating of the intensity and frequency of the peripheral nerve stimulation comprises:
segmenting the stimulation into a constant burst period on time and a variable burst period off time;
modulating the intensity of the stimulation during the burst period on time; and
modulating the duration of the burst period off time.

16. The method of claim 1, wherein the subject is a human being.

17. The method of claim 1, wherein the sensory feedback comprises a perceived frequency of a percept, and a perceived intensity of a percept.

18. The method of claim 1, wherein the sensory feedback is received from one or more sensors.

19. The method of claim 1, wherein the method is repeated until optimized peripheral nerve stimulation parameters are identified.

20. The method of claim 1, wherein the method occurs through the use of a peripheral nerve stimulation (PNS) sensory feedback system

21. The method of claim 1, wherein the method is used to enhance nerve sensations by the subject, enhance sensory feedback from prosthetic limbs, enhance sensory feedback from brain computer interfaces (BCI), enhance the operation of sensory therapeutic orthoses, enhance the effectiveness of neuromodulation therapies, or combinations thereof.

22. A system for optimizing peripheral nerve stimulation in a subject, comprising:
a memory; and
at least one processor coupled to the memory and configured to implement a method, the method comprising:
stimulating a peripheral nerve of the subject, wherein the stimulation comprises modulating an intensity and frequency of the stimulation, and
receiving sensory feedback from the subject after the stimulation.

23. The system of claim 22, wherein the modulating comprises modulating an intensity and frequency of a received percept of the stimulation.

24. The system of claim 22, wherein the system further comprises one or more electrodes, wherein the one or more electrodes are operable to be associated with or near the peripheral nerve, and wherein the one or more electrodes are operable to provide peripheral nerve stimulation.

25. The system of claim 22, wherein the modulating of the stimulation comprises adjusting stimulation current around a reference point.

26. The system of claim 22, wherein the modulating of the stimulation comprises segmenting the stimulation into on and off cycles.

27. The system of claim 26, wherein the modulated frequency comprises a stimulation burst period comprising an on time and an off time.

28. The system of claim 27, wherein the modulating of the intensity and frequency of the peripheral nerve stimulation comprises:
segmenting the stimulation into a burst period on time and a burst period off time;
modulating the intensity of the stimulation during the burst period on time; and
modulating the duration of the burst period off time, burst period on time, or combinations thereof.

29. The system of claim 28, wherein the modulating of the intensity and frequency of the peripheral nerve stimulation comprises:
segmenting the stimulation into a constant burst period on time and a variable burst period off time;
modulating the intensity of the stimulation during the burst period on time; and
modulating the duration of the burst period off time.

30. The system of claim 22, further comprising one or more sensors operable to receive the sensory feedback.

31. The system of claim 22, wherein the system is in the form of a peripheral nerve stimulation (PNS) sensory feedback system.

32. A computing device comprising a non-transitory computer-usable medium having computer-readable program code embodied therein, the computer-readable program code adapted to be executed to implement a method for optimizing peripheral nerve stimulation in a subject, wherein the method comprises:
stimulating a peripheral nerve of the subject, wherein the stimulation comprises modulating an intensity and frequency of the stimulation; and
receiving sensory feedback from the subject after the stimulation.
